(19) United States
(12) Patent Application Publication
(10) Pub. No.: US 2011/0129553 A1
(43) Pub. Date: Jun. 2, 2011

(54) PROCESS FOR THE PREPARATION OF SOUR CHERRY SEED EXTRACT, USE OF THE EXTRACT FOR THE PREPARATION OF PHARMACEUTICAL COMPOSITIONS AND PHARMACEUTICAL COMPOSITIONS CONTAINING SAID EXTRACT

(75) Inventors: Árpád Tósaki, Debrecen (HU); Miklós Vecsernyés, Debrecen (HU); László Fésüs, Debrecen (HU); István Bak, Debrecen (HU); Béla Juhász, Debrecen-Jozsa (HU); László Papp, Fehergyarmat (HU); Szilvia Tóth, Debrecen (HU)

(73) Assignee: Debreceni Egyetem, Debrecen (HU)

(21) Appl. No.: 13/008,147

(22) Filed: Jan. 18, 2011

Related U.S. Application Data
(62) Division of application No. 12/091,440, filed on Apr. 24, 2008, now abandoned.

(30) Foreign Application Priority Data
Oct. 25, 2005 (HU) P0500975

(51) Int. Cl. A61K 36/736; A61K 31/353; A61K 31/7048; A61P 9/00; A61P 9/10

(52) U.S. Cl. 424/735; 514/456; 514/27

(57) ABSTRACT

The present invention relates to a process for the isolation of the components of seed of Primus cerasus (sour cherry), the components thus obtained, pharmaceutical compositions containing said components as well as the use of the components for the preparation of cardioprotective pharmaceutical compositions. The components are especially useful for improving circulation, preventing stenosis or ameliorating ischemia-induced myocardial damages.

### TECHNICAL FIELD

The present invention relates to a process for the isolation of the components of seed of Prunus cerasus (sour cherry), the components thus obtained, pharmaceutical compositions containing said components as well as the use of the components for the preparation of cardioprotective pharmaceutical compositions.

### BACKGROUND ART

Disorders of the cardiovascular circulation are major causes of morbidity and mortality and can result in life-long disabilities in survivors. For the 13 million people worldwide affected by heart failure and nearly 1,000 individuals succumb to sudden cardiac death in the US each day as a result of fatal ventricular arrhythmias (Pearson, 2004; Ackerman, 2004). Most of sudden deaths claim middle-aged and elderly populations. Therefore, the high morbidity and mortality of cardiovascular diseases have focused the attention of physicians and clinicians on restoring coronary blood flow to resuscitate the ischemic or hypoxic myocardium. The appropriate pharmacological interventions and therapy can facilitate the salvage of myocardium, improve cardiac function, and decrease cardiac morbidity and mortality.

According to the above there is a need for active substances, Particularly there is a need for active substances of natural origin.

### DISCLOSURE OF THE INVENTION

According to the above, the aim of the present invention is to prepare active substances—possibly of natural origin—which successfully prevent, improve or reverse the above disorders and conditions.

According to the present invention the aim is achieved by obtaining said active substances from the components of sour cherry seed.

Thus, the present invention relates to a process for the preparation and isolation of the components of sour cherry seed, and the components thus obtained.

The invention relates further to the use of said components for the preparation cardioprotective pharmaceutical compositions.

The invention also relates to pharmaceutical compositions comprising the components prepared according to the process of the invention.

There are several prior art documents discussing the use of various components of sour cherry, however, no document can be found which discloses the isolation of the components of sour cherry seed, or suggest the use of the same for the treatment and/or prevention of cardiac disorders.

Surprisingly, we found that the sour cherry seed extract exerts cardioprotective activity in various biological samples. As an outstanding result, the tests sowed that the extract used do not involves any side effects.

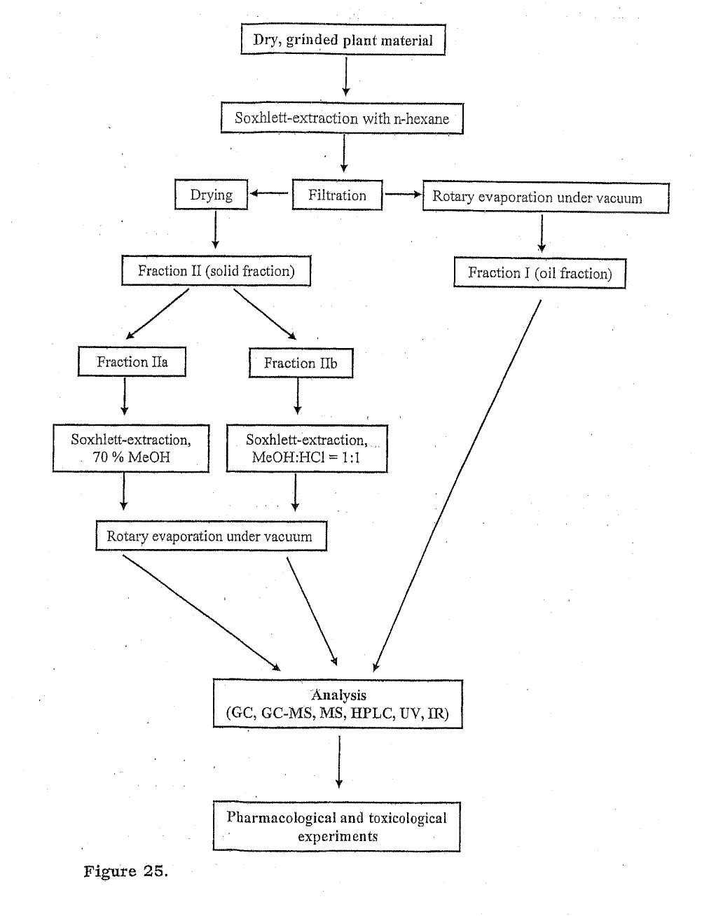

According to the present invention, the process of the invention, after removing the wall of seed, leads to Fraction I (oil fraction) and Fraction II (solid phase) of Prunus cerasus (sour cherry) seed. The steps of separation are depicted in FIG. 25.

The invention features cardioprotective effects with no adverse effects of sour cherry (Prunus cerasus) seed extract in biological samples.

The sour cherry seed contains two main fractions:

Fraction I: The sour cherry seed contains 35% of oil fraction (O) including vitamin E (alpha-tocopherol, 52 mg/100 g), vitamin E-like components (delta-tocopherol, tocotrienol), unsaturated free fatty acid esters (hexa-, hepta-, and octadecane acids, aldehide (e.g., hexanal), mixtures of triglycerids including free fatty acids LLL (L: linoleic acid) LLO (O: oleic acid), LLP (P: palmitil acid). The total tocopherol content of the O fraction of sour cherry seed is about 90 mg/100 g. The O fraction does not contain flavonoids, polyphenols, and cyanide components in comparison with the Fraction II (see below).

Fraction II: the solid (S) fraction of sour cherry seed includes flavonoids, rhamnetin, malvidin, delfinidin, pinocembrin, naringenin, quercetin, rezveratrol, dihydroquercetin, peonidin, apigenin, pro- and athocyanidins, glucose (e.g., feruloil-D-glucose, cumaroil-glucose, feruloil-d-glucose), stilbenes, catechins, gallic acid, gallocatechins, and other antioxidants (e.g., gallotannin). The fraction II was divided in two Parts (fraction IIa and fraction IIb) according to the extraction procedure of sour cherry seeds. Thus, fraction IIa was obtained with the extraction of 70% of methanol, and fraction IIb was the product of seed extraction using methanol and hydrochloric acid mixture (9:1).

### RESULTS

### Analysis of Sour Cherry (Prunus cerasus) Seed

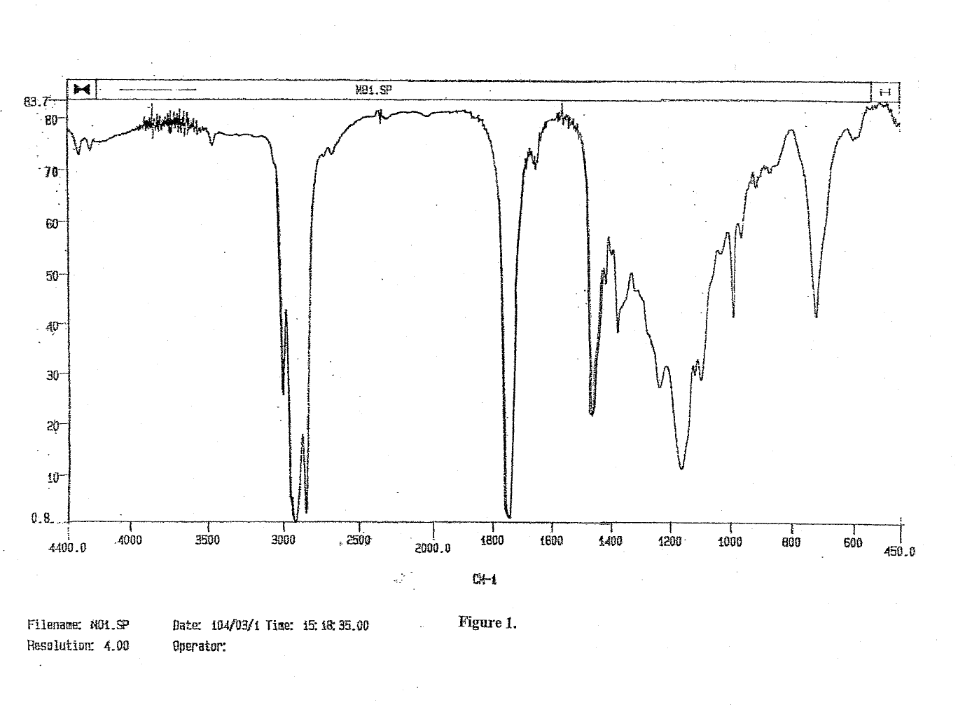

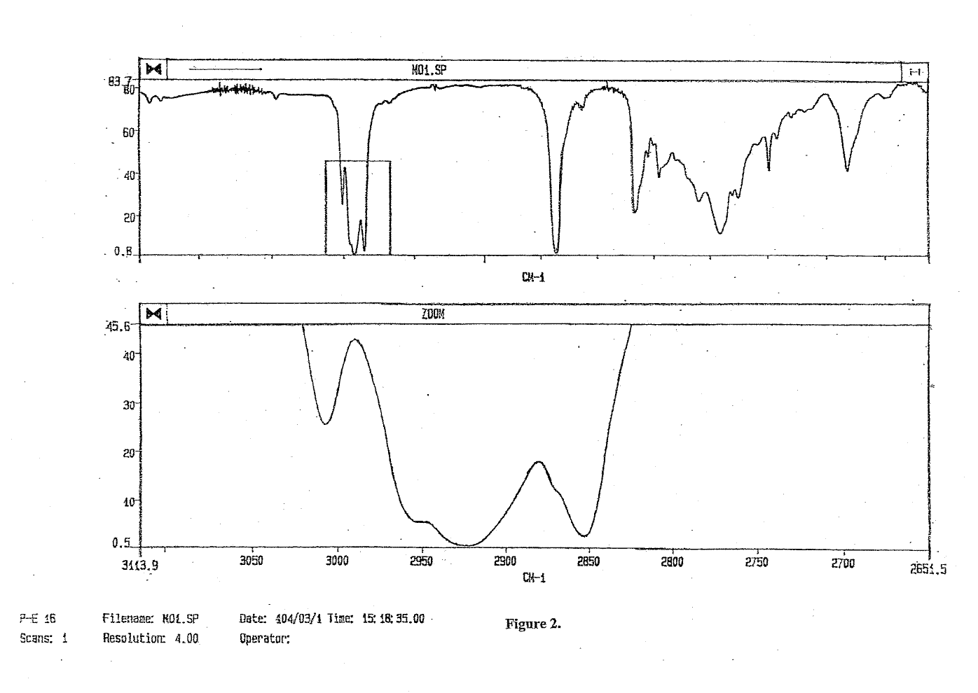

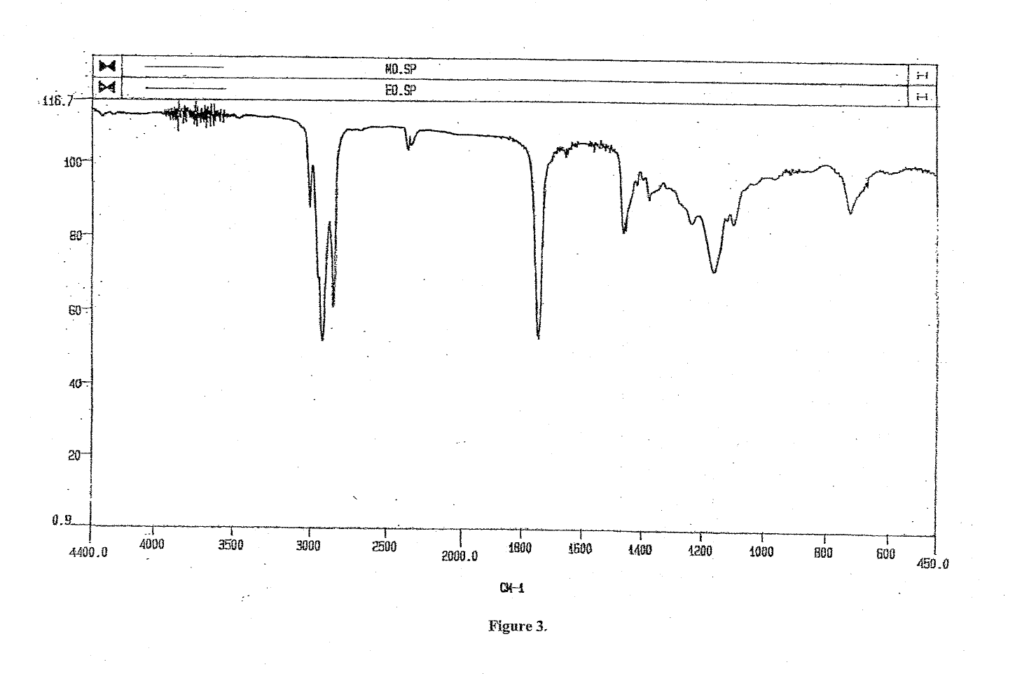

FIG. 1, FIG. 2, and FIG. 3 show the infra red (IR) spectra of O fraction. FIG. 1 shows a typical unsaturated fatty acid ester component at 3020 cm⁻¹. An ester group (═O) can be detected at 1742 cm⁻¹ of the spectra. Between 2500 and 2800 cm⁻¹, OH⁻ group peaks are detected indicating the components of free carbonyl acids. The long carbonyl chain components can bee seen at the ranges between 1460 and 720 cm⁻¹, and 3000 and 2800 cm⁻¹

The spectra of O of sour cherry seed was compared to the sunflower's oil (FIG. 3) and many similarities were found. However, the major difference between the O fraction of the sour cherry seed and sunflower's oil is in the content of free fatty acids. Thus, free fatty acids can not be found (or in a very little amount) in the sunflower's oil. However, the O fraction of the sour cherry seed contains a relatively high amount of free fatty acids detected between 970 and 930 cm⁻¹.

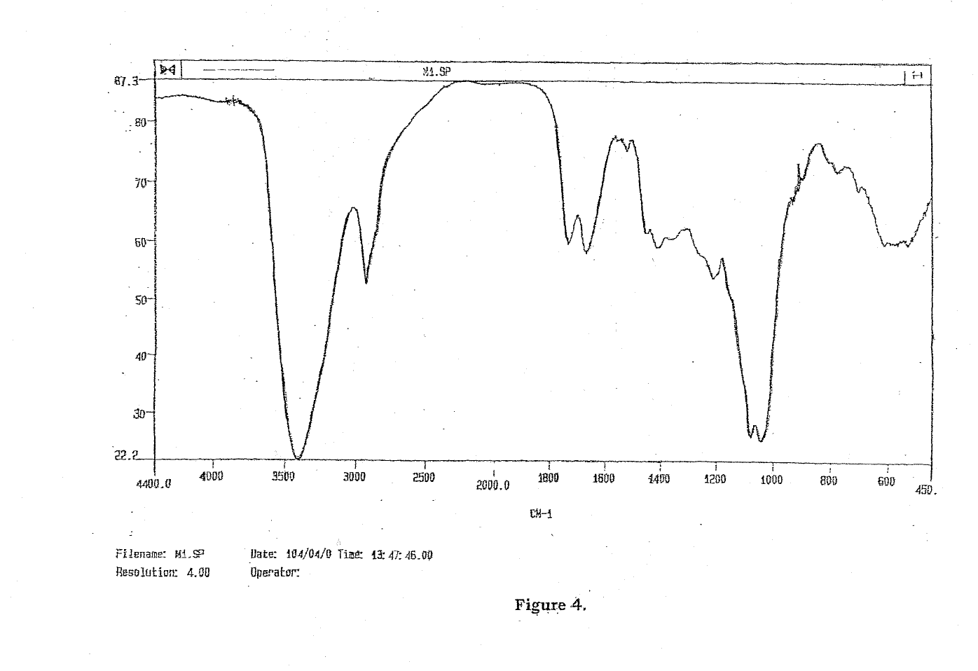

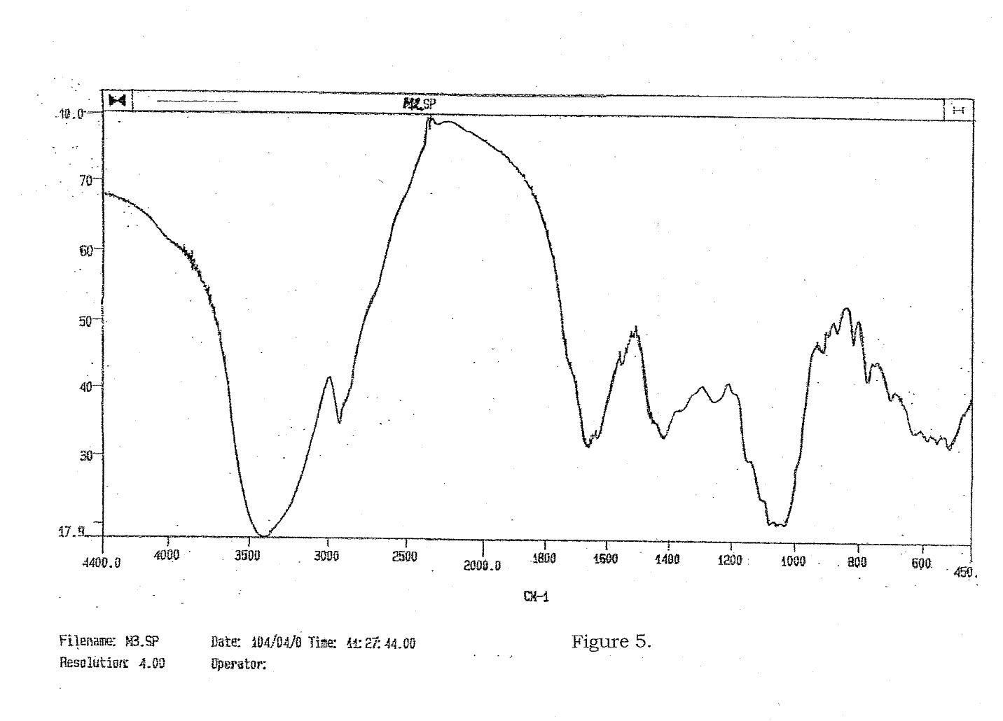

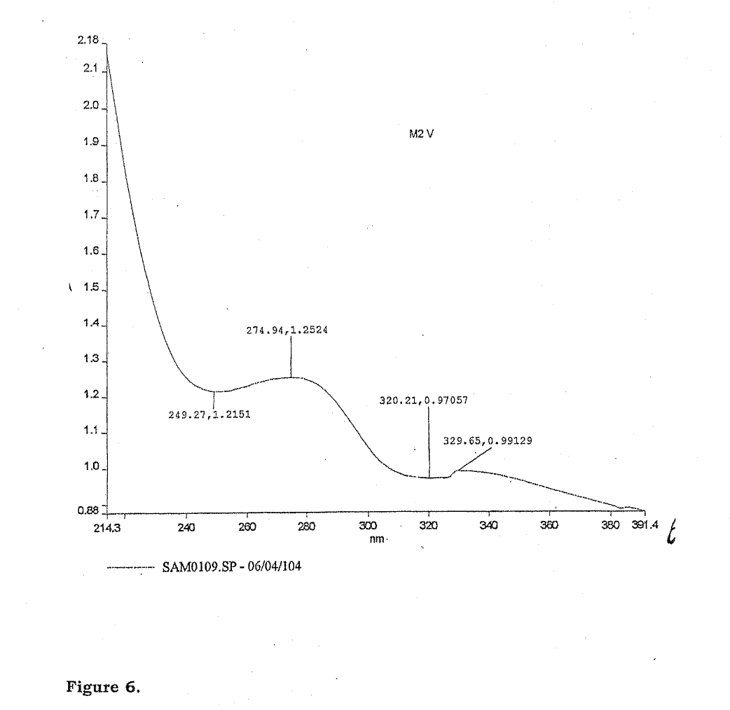

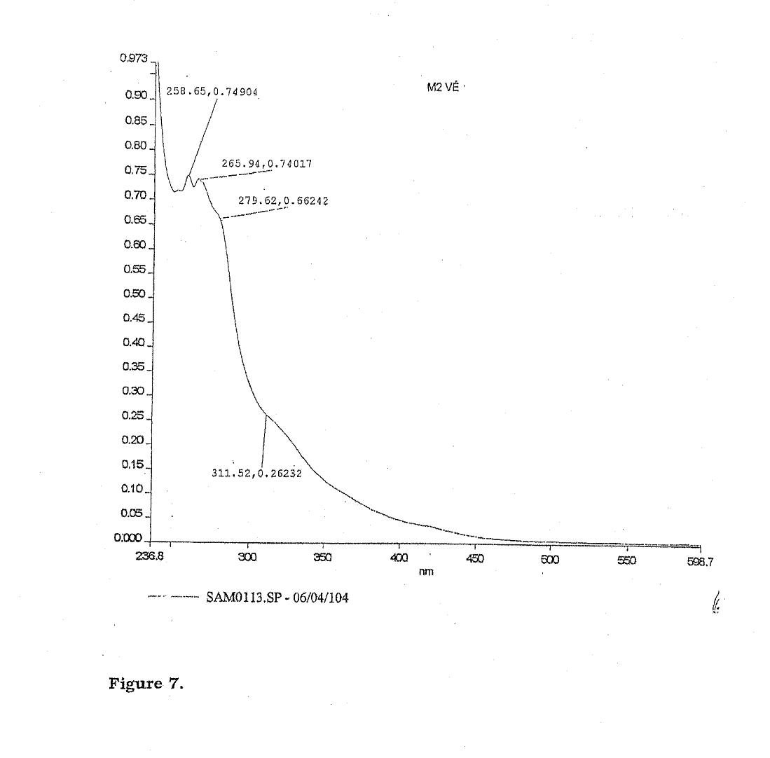

The IR spectra of fractions IIa and IIb could be seen in FIG. 4 and FIG. 5. It is well shown that fraction IIa contains ester components indicating by the peek at 1666 cm⁻¹ (carbonyl component). The peaks of IR spectra, at 3400 and 1050 cm⁻¹, indicates a substantial numbers of hydroxyl groups. The fraction IIb does not contain ester components, and this is the so called flavonoid-fraction. This is proven by the UV spectra in FIG. 6 showing the peaks at 330 nm and 275 nm, respectively. The UV absorbance spectra of the fraction IIb at 430 nm (FIG. 7) indicates the presence of anthocyan and proanthocyanidin components which is proven by the red color of the extract.

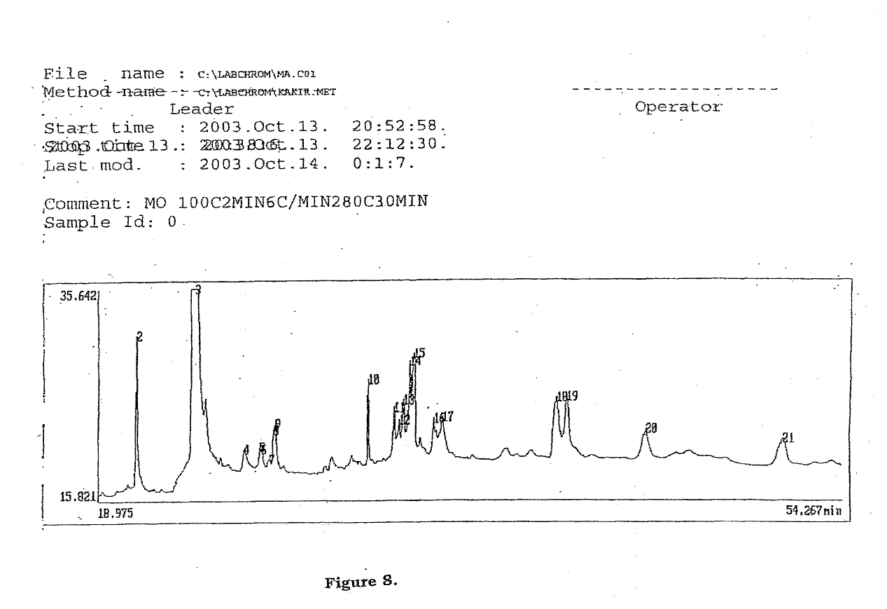

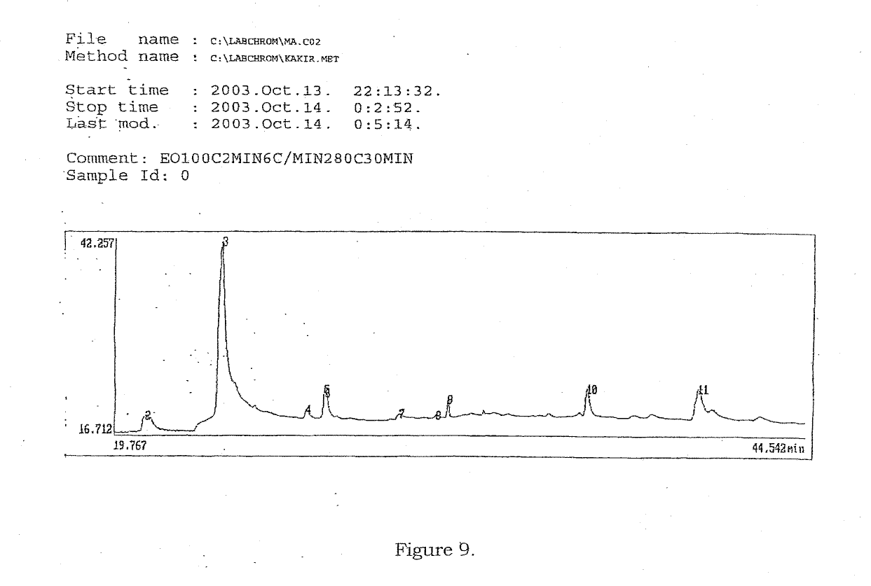

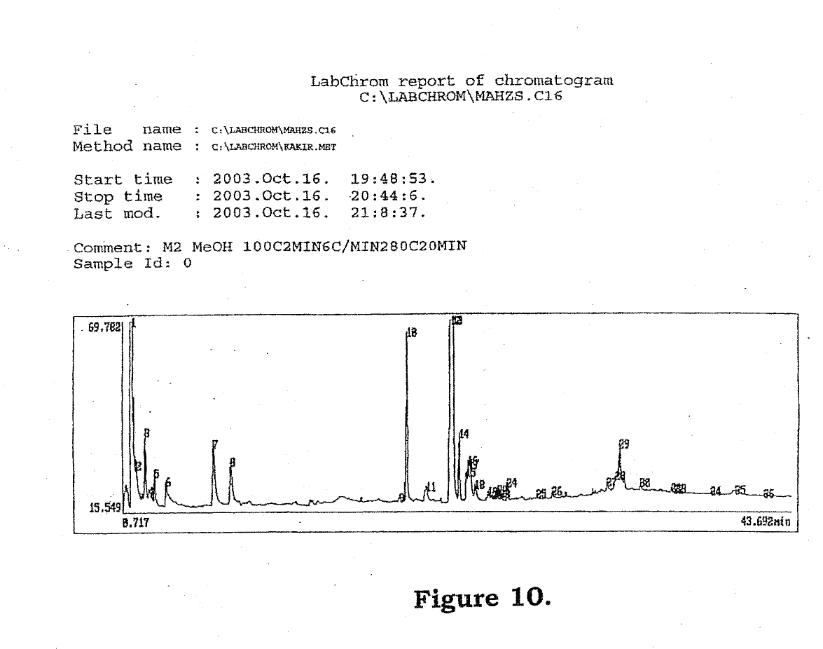

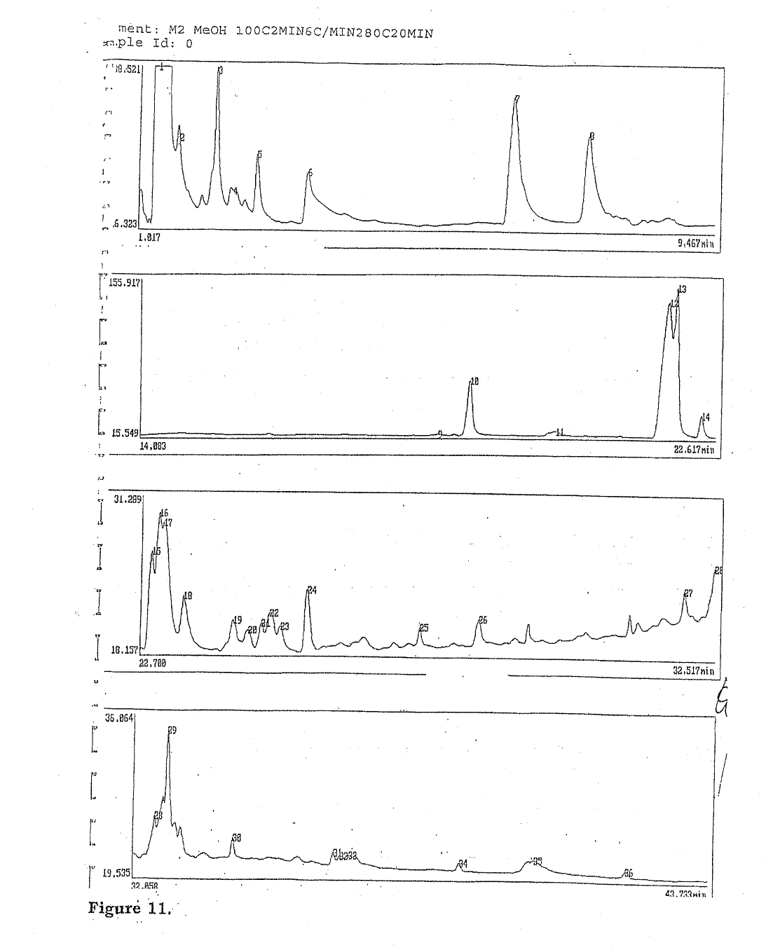

FIG. 8 shows the gas chromatogram (GC) of O fraction in comparison with the sunflower (FIG. 9) chromatogram. The O fraction of sour cherry seed extract, beside the main components, contains many minor components (FIG. 8) in comparison with the analysis of sunflower oil (FIG. 9). The fraction IIb (solid fraction) also contains volatile components (FIG. 10). FIG. 11 shows the GC results in detail obtained from FIG. 10. The O fraction does not contain organic-cyanide components, however, fraction IIa contains cyanide components like amygdaline. Polyphenols and flavonoids cannot be detected in O fraction. These components (polyphenols and flavonoids) are detected in fractions IIa and IIb. The so-called Folin-Ciocelteau method indicates that fraction IIb has gallic acid-like components about 205.6 mg gallic acid components (polyphenols) in 100 g sour cherry seed extract.

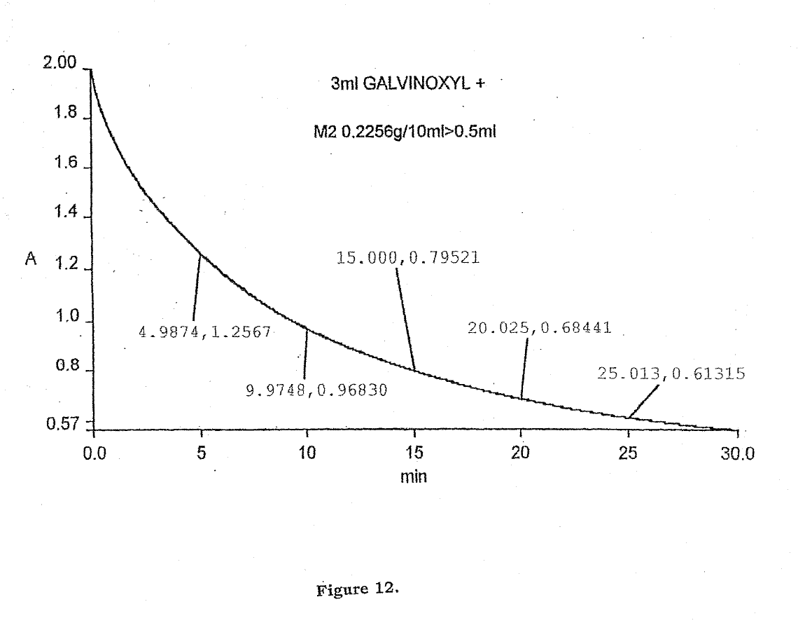

The free radical scavenger activity of each sample (O, fraction IIa and IIb) was studied by galvinoxyl radical method. The results show that the O fraction and fraction IIb possess free radical scavenger activities. The use of galvinoxyl technique (UV study) indicates (FIG. 12) that fraction IIb in the presence of alcohol showed UV absorption, directly supporting that fraction IIb contains flavonoids.

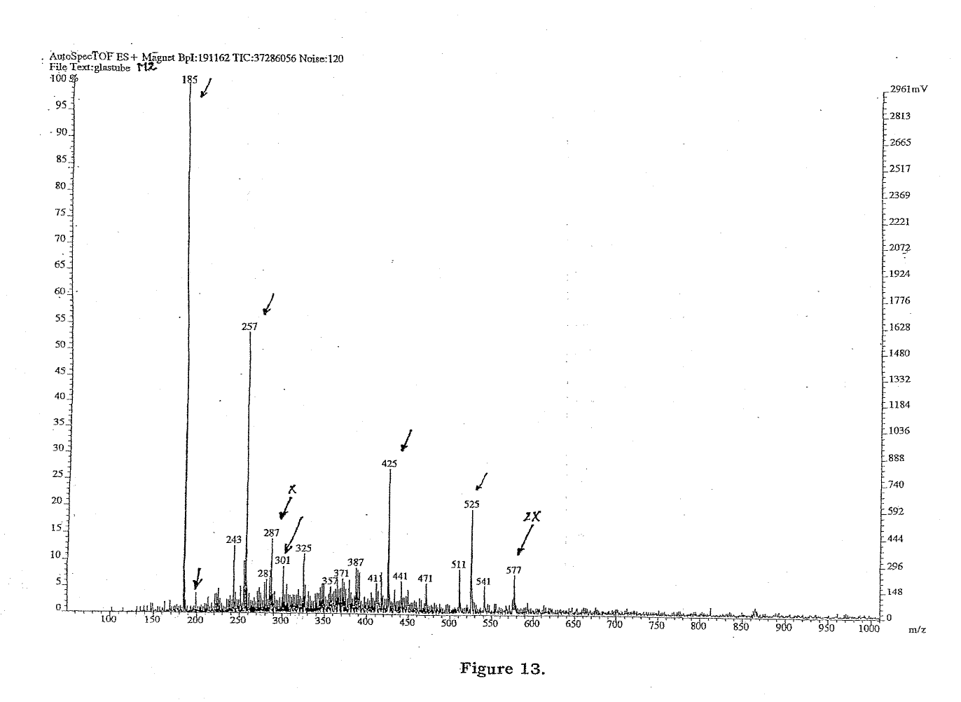

FIGS. 13-17 show the analysis of Prunus cerasus seed by mass spectroscopy (MS). Thus, the fraction IIb (FIG. 13) contains dihydro-p-cumaric acid indicating by the peak at 185 m/z (M+1), ferrulic acid at 213 m/z (M+1) and, and this latter peak is overlapped by the peek of coffee acid at 213 m/z (M+1) as well. Major components of fraction II are cyanidin at 287 m/z and peonidin at 301 m/z. The peak at 301 m/z (M−1) proofs the presence of quercetin in fraction IIb. Furthermore, there is a peak of dimmer cyanidin (procyanidin) at 577 m/z giving a light/red color of the extract. The typical flavon components are pinocembrin at 257 m/z M+H), and tangeretin at 371 m/z, respectively. The peaks between 425 and 525 m/z suggest the presence of vitamin E-like compounds in fraction IIb.

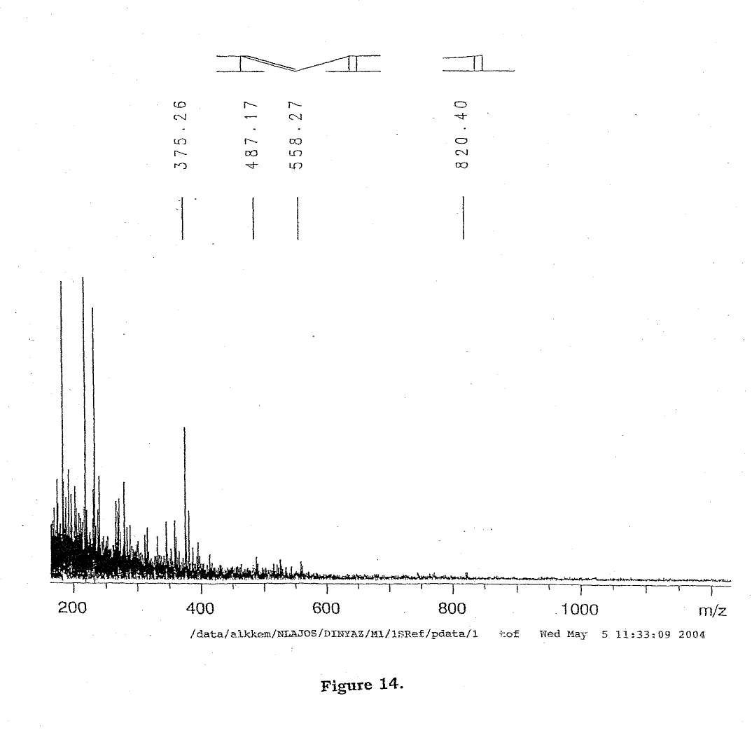

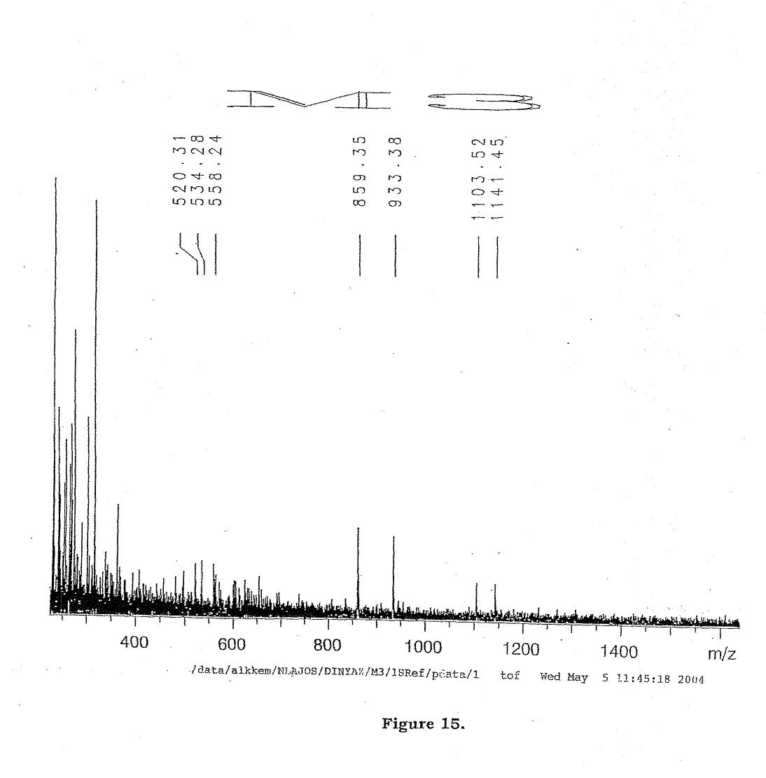

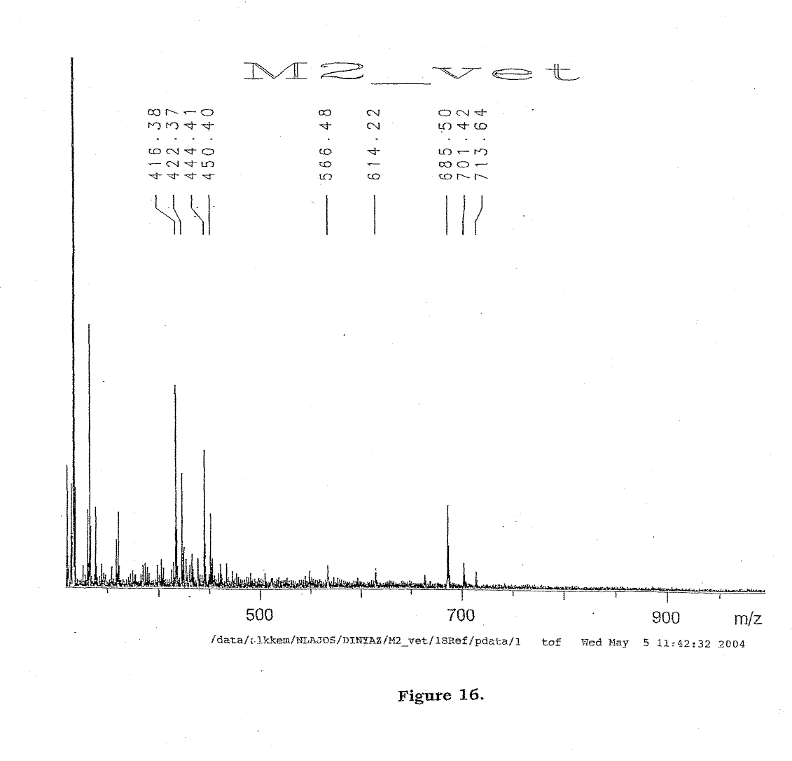

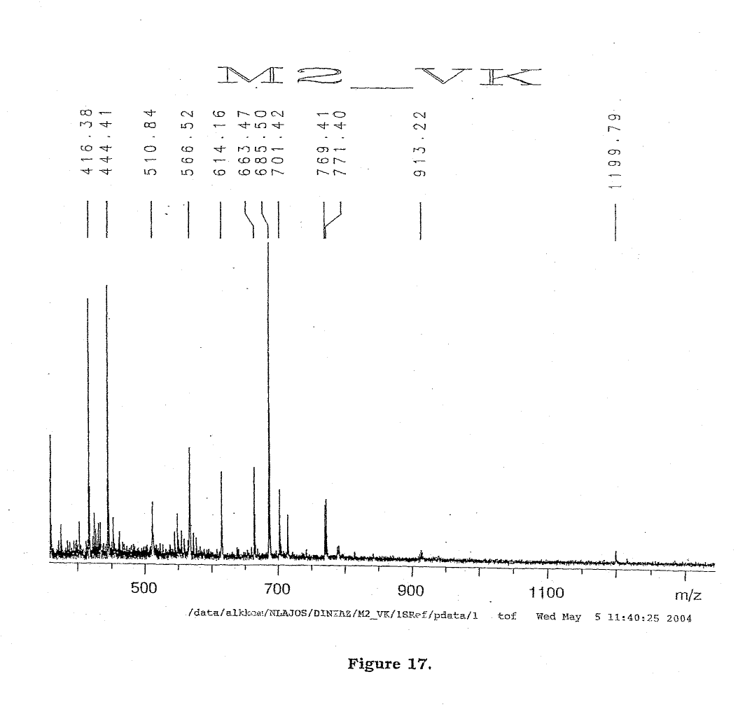

The use of MALDI-TOF spectra analysis shows some high molecular weight components. Thus, the peak at 487 m/z indicates the presence of quercetin-3-glucosid M+Na) in fraction IIa (FIG. 14). Furthermore, the peak at 820 m/z (M+H₂O) is corresponding with the chlorogen acid-related acetylated quercetin-3-glucosid compound (FIG. 14). The peak appeared at 1141 m/z shows the presence of gallic acid-related acetylated procyanidin trimer (M+Na) (FIG. 15). The peak of epicatechin-3-gallate dimer form is appeared at 859 m/z (FIG. 15). Acetylated form of catechin-3-glucoside by cumarin acid is detected at 685 m/z (FIG. 16). FIG. 17 shows the presence of galangin acetylated by p-cumarin acid at 685 m/z. Total flavonoid concentrations of fractions IIa and IIb are about 2%.

GC-MS Studies:

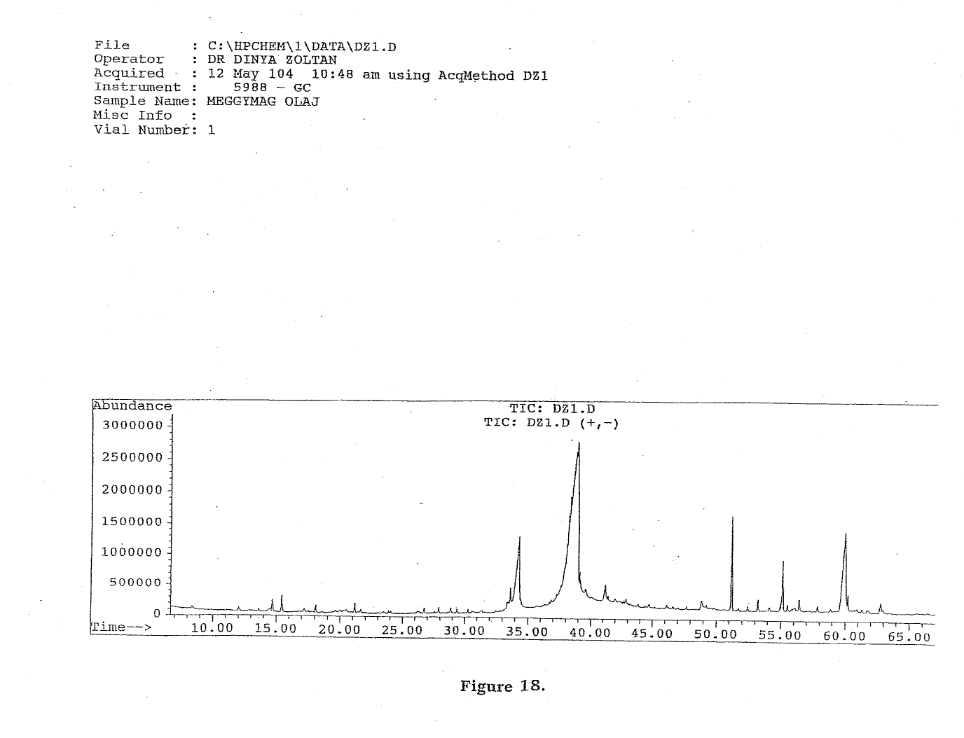

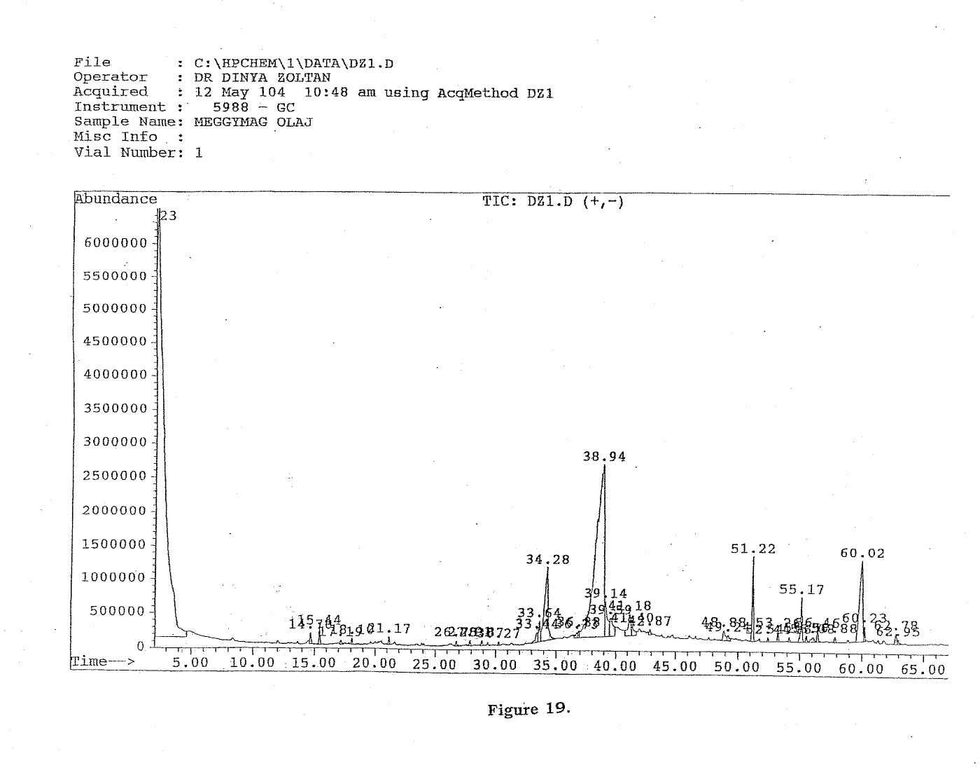

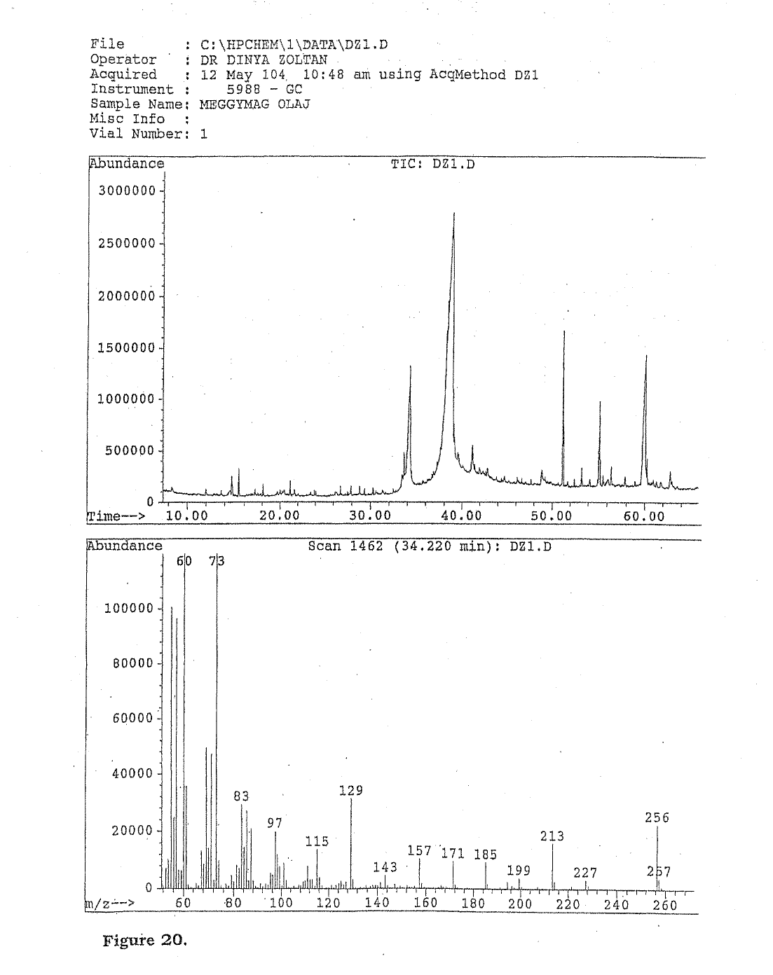

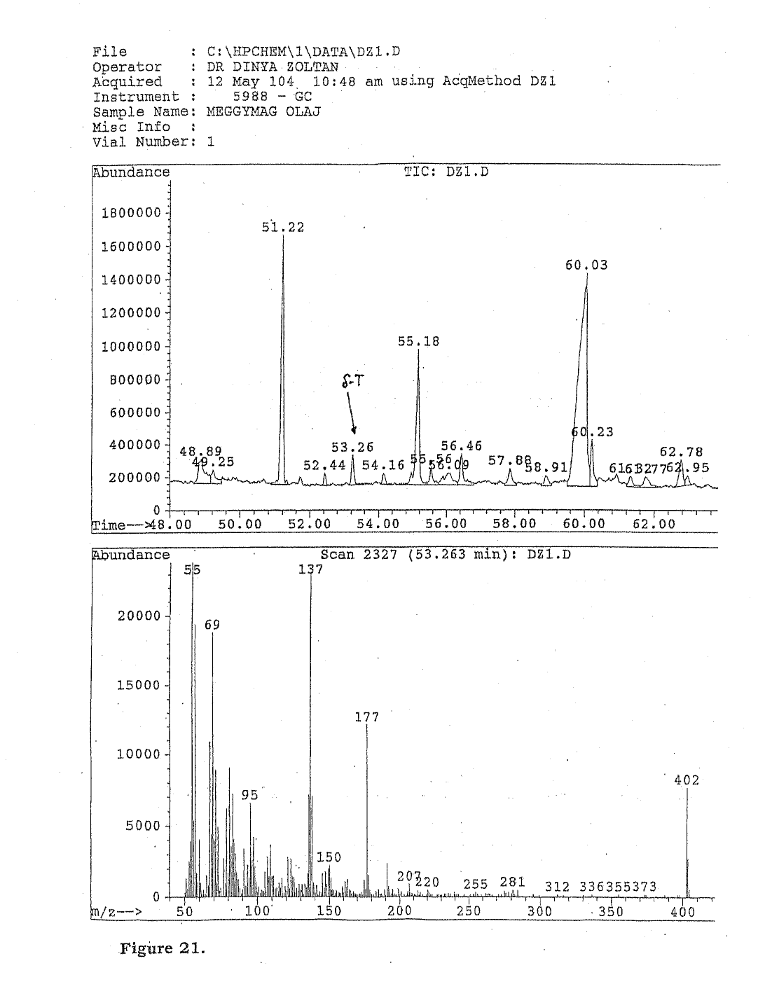

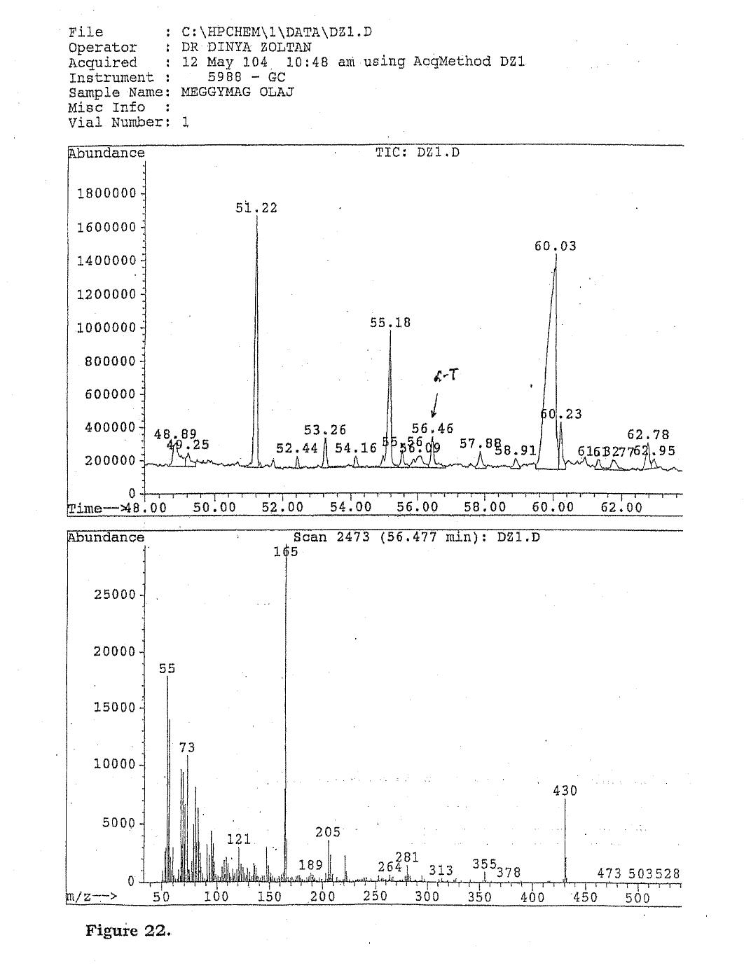

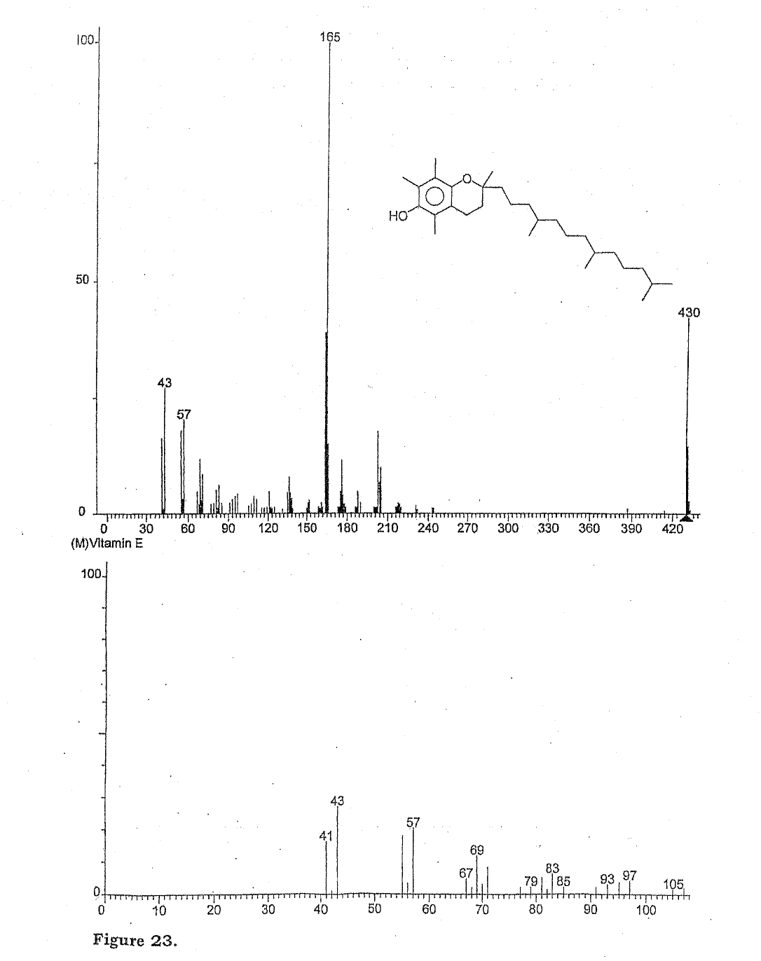

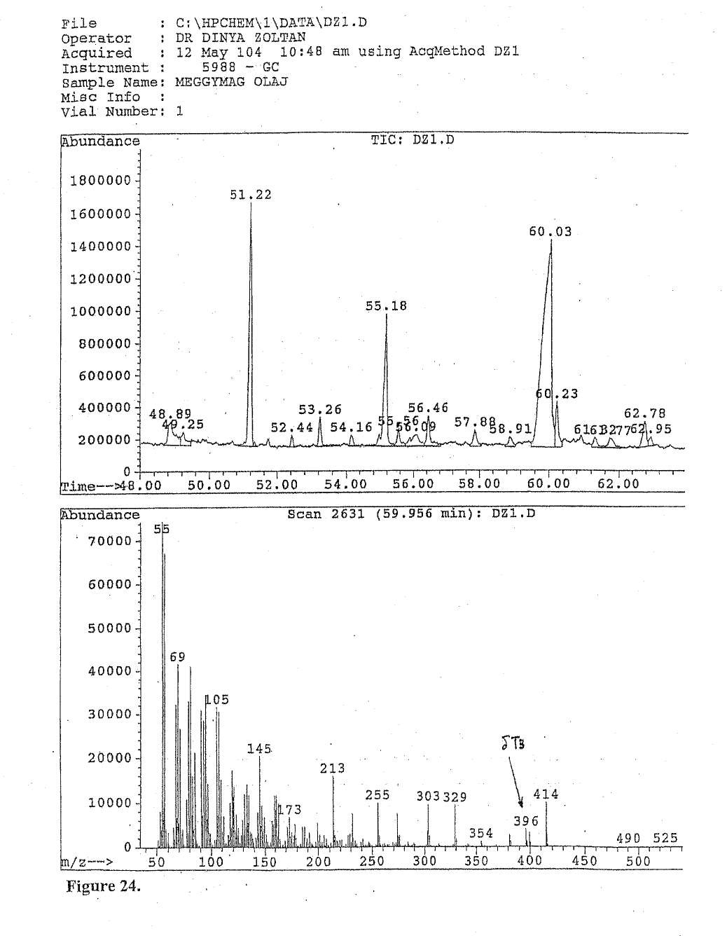

Chromatograms were obtained by total ion chromatography. FIGS. 18 and 19 show that ‘O’ fraction consists of mainly triglycerides including linoleic acid (LA), oleic acid (OL). However, a small amount of palmitil acid and stearin acid was also detected. Thus, the ‘O’ fraction contains mainly unsaturated triglyceride components. Beside triglycerides, free fatty acids such as ω-3 α-linoleic acid, hexa-, hepta-, octadecanoic acids, and aldehydes (e.g., hexanal and decadienal) can also be detected in the ‘O’ fraction (FIG. 20). The most important components of the ‘O’ fraction are vitamin E and its isomers (FIGS. 21-24). Thus, δ-tocopherol (FIG. 21), α-tocopherol (FIGS. 22 and 23), and δ-tocotrienol (FIG. 24) are the major components. The α-tocopherol content is 52-53 mg/100 g, while the total tocopherol content is 80-85 mg/100 g.

### Pharmacological Effects of the Sour Cherry (Prunus Cerasus) Seed Extract (Fractions I and II)

### Methods:

### (i) Isolated Rat and Mouse Heart Preparations:

Male Sprague-Dawley rats (320-350 g) were used for all studies. Animals received humane care in compliance with the “Principles of Laboratory Animal Care” formulated by the National Society for Medical Research prepared by the National Academy of Sciences (Publication No. 86-23, revised 1985). Rats were anesthetized with i.p. pentobarbital (60 mg/kg) and then given intravenous heparin (500 IU/kg). After thoracotomy, the heart was excised, and the aorta and left atrium were cannulated. Hearts were initially perfused according to Langendorff then preparations were switched to the working mode as previously described (Tosaki and Braquet, 1990). The isolated mouse heart preparation was carried out as described by Bak et al (2003).

### (ii) Experimental Time Course and Idices Measured:

Before the onset of ischemia and reperfusion, and the isolation of hearts, rats and mice were treated orally with various doses (1 mg/kg/day, 5 mg/kg/day, 10 mg/kg/day, and 30 mg/kg/day) of the sour cherry seed extract (the components of fraction IIa and fraction IIb), respectively, for 14 days.

The extract of sour cherry seed (fractions IIa and IIb) was homogenized in 2 ml of 1% methylcellulose solution and then diluted with 0.9% of NaCl to 10 ml. Rats were orally treated daily with 10 ml/kg of the solution (containing 1 mg/kg, 5 mg/kg, 10 mg/kg or 30 mg/kg of flavonoid-rich extract, fractions IIa and IIb) together) for 14 days, and no changes in the behavior and physical activities of animals were observed during the treatment. After 14 days pretreatment, hearts were isolated and subjected to 30 min of ischemia followed by two hours of reperfusion.

An epicardial ECG was recorded by a computer acquisition system throughout the experimental period by two silver electrodes attached directly to the heart. The ECGs were analyzed to determine the incidence of VF and VT. Hearts were considered to be in VF if an irregular undulating baseline was apparent on the ECG. VT was defined as five or more consecutive premature ventricular complexes, and this classification included repetitive monomorphic VT which is difficult to dissociate from rapid VT. The heart was considered to be in sinus rhythm if normal sinus complexes occurring in a regular rhythm were apparent on the ECG. Aortic flow was measured by an in-line flow rotameter. Coronary flow rate was measured by a timed collection of the coronary effluent that dripped from the heart. Before ischemia and during reperfusion, heart rate (HR), coronary flow (CF) and aortic flow (AF) rates were registered. Left ventricular developed pressure (LVDP) was also recorded by the insertion of a catheter into the left ventricle via the left atrium and mitral valve. The hemodynamic parameters were registered by computer acquisition system (PouwerLab, ADInstruments, Australia).

Determination of Infarct Size:

Hearts for infarct size measurement were perfused, at the end of each experiment, with 25 ml of 1% triphenyl tetrazolium solution in phosphate buffer (Na₂HPO₄ 88 mM, NaH₂PO₄ 1.8 mM) via the side arm of the aortic cannula, then stored at −70° C. for later analysis. Frozen hearts were sliced transversely (Schultz et al., 1997) in a plane perpendicular to the apico-basal axis into 2-3 mm thick sections, weighted, blotted dry, placed in between microscope slides and scanned on a Hewlett-Packard Scanjet 5p single pass flat bed scanner (Hewlett-Packard, Palo Alto, Calif.). Using the NIH Image 1.61 image processing software, each digitalized image was subjected to equivalent degrees of background subtraction, brightness and contrast enhancement for improved clarity. Infarct zones of each slice were traced and the respective areas were calculated in terms of pixels (Dickson et al., 2001). The areas were measured by computerized planimetry software and these areas were multiplied by the weight of each slice, then the results summed up to obtain the weight of the risk zone (total weight of the left ventricle, mg) and the infarct zone (mg). Infarct size was expressed as the ratio, in percent, of the infarct zone to the risk zone.

Measurement of Caspase III Activity by Iummunocytochemistry:

The free-floating sections of the heart were first incubated with biotinylated goat anti-caspase-3 antibody (Sigma, St. Louis, Mo., USA; diluted 1:1000) for 2 days at 4° C. The immunological and immunocytochemical characteristics of antibody have been published earlier (Hatib-Al-Khatib et al., 2004). The sections were then transferred into a solution of biotinylated goat antirabbit (Vector Laboratories, Burlingame, Calif., USA; diluted 1:200) for 50 min at room temperature, than avidin-biotinylated-peroxidase complex (ABC; Vector Laboratories, Burlingame, Calif., USA; diluted 1:100) for 4 h at room temperature, and was completed with a diaminobenzidine chromogen reaction (Hancock, 1984). Prior to the antibody treatments sections were kept in 10% normal goat serum (Vector Laboratories, Burlingame, Calif., USA) for 50 min, All incubations were performed under continuous gentle agitation, and all of antibodies were diluted in 10 mM phosphate-buffered saline (PBS, pH 7.4) to which 0.1% Triton X-100 and 1% normal rabbit serum (Vector Laboratories, Burlingame, Calif., USA) were added. Sections were mounted on gelatin-coated slides and covered with Permount neutral medium (Fluka, Buchs, Switzerland).

Statistics:

The data for HR, CF, AF, LVDP, caspase-3 activity; and infarct size were expressed as the mean±SEM. One-way analysis of variance test was first carried out to test for any differences between the mean values of all groups. If differences were established, the values of sour cherry seed extract (fractions IIa and IIb together) treated groups were compared with those of the drug-free control group by multiple t-test followed by Bonferroni correction. For the distribution of discrete variables such as the incidence of VF and VT which follows a nonparametric distribution, an overall chi-square test for a 2×n table was constructed followed by a sequence of 2×2 chi-square tests to compare individual groups. A change of p<0.05 between the drug-free control and treated groups was considered to be significant.

### Results (Pharmacological Studies):

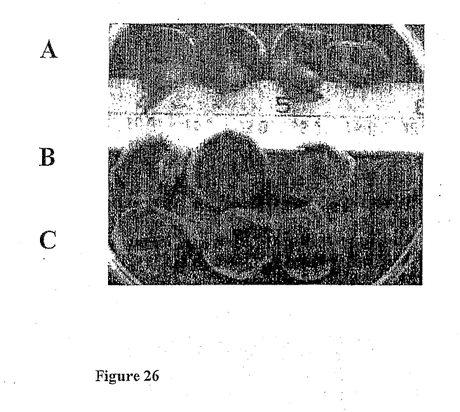

FIG. 26 shows the representative picture of Prunus cerasus (sour cherry) seed extract (10 mg and 30 mg/kg) on infarct size limitation in isolated rat hearts subjected to 30 min of ischemic followed by 120 min of reperfusion. White areas represent infracted areas. FIG. 26A shows infarct size in the drug-free ischemic/reperfused myocardium, and FIG. 26B and FIG. 26C show infarct size in hearts treated with 10 mg and 30 mg/kg of sour cherry seed extract (fractions IIa and IIb together), respectively.

Table 1 (below) shows the numerical (in each heart) values of infarct size in hearts (n=6 in each group) obtained from rats treated with various doses of sour cherry seed extract (fractions IIa and IIb together) for 14 days, and subjected to 30 min of ischemia followed by 120 min of reperfusion. The incidence of VF and VT were also detected (n=12 in each group). Comparisons were made to the values of the drug-free ischemic/reperfused control group. *p<0.05. Thus, in hearts treated with 10 mg/kg and 30 mg/kg of sour cherry seed extract, a significant reduction in the infarct size, the incidence (%) of VF, and the incidence (%) of VT were reduced from their drug-free control values of 38.3%±1.3% (infarct size), 93% (VF), and 100% (VT) to 26.5%±2% (infarct size, *p<0.05, 10 mg/kg sour cherry) and 21.8%+1.8% (infarct size, *p<0.05, 30 mg/kg sour cherry), 50% (VF, 10 mg/kg sour cherry) and 17% (VF, 30 mg/kg sour cherry, *p<0.05), and 58% (VT, 10 mg/kg sour cherry) and 25% (VT, 30 mg/kg sour cherry, *p<0.05), respectively.

TABLE 1
Effect of sour cherry seed extract (fractions IIa and IIb together)
on infarct size, and incidence (%) of VT and VF. Each
individual value is shown, and comparisons were made to the
values of the drug-free control (Table 1A) group.

| | | | |
|---|---|---|---|
| No. of | Infarct size | Incidence (%) | Incidence (%) |
| Hearts | (%) | of VF | of VT |

A). Drug-free control group: rats were orally treated with vehicle for
14 days then hearts were isolated and subjected to 30 min ischemia
followed by 120 min reperfusion.

| | | | |
|---|---|---|---|
| 1. | 40 | + | + |
| 2. | 34 | − | + |
| 3. | 44 | + | + |
| 4. | 38 | + | + |
| 5. | 35 | + | + |
| 6. | 39 | + | + |
| 7. | | + | + |
| 8. | | + | + |
| 9. | | + | + |
| 10. | | + | + |
| 11. | | + | + |
| 12. | | + | + |
| mean | 38.3 | 93% | 100% |
| SD | 3.3 | | |
| SE | 1.3 | | |

B). Rats were orally treated with 10 mg/kg of sour cherry seed extract
(fractions IIa and IIb together) for 14 days then hearts were isolated
and subjected to 30 min ischemia followed by 120 min reperfusion.

| | | | |
|---|---|---|---|
| 1. | 28 | + | + |
| 2. | 22 | − | + |
| 3. | 30 | + | + |
| 4. | 35 | + | + |
| 5. | 21 | + | + |
| 6. | 23 | − | − |
| 7. | | − | − |
| 8. | | − | − |
| 9. | | − | − |
| 10. | | + | + |
| 11. | | + | + |
| 12. | | − | − |
| mean | 26.5* | 50% | 58% |
| SD | 5.0 | | |
| SE | 2.0 | | |

C). Rats were orally treated with 30 mg/kg of sour cherry seed extract
(fractions IIa and IIb together) for 14 days then hearts were isolated
and subjected to 30 min ischemia followed by 120 min reperfusion.

| | | | |
|---|---|---|---|
| 1. | 23 | + | + |
| 2. | 28 | − | − |
| 3. | 19 | − | − |
| 4. | 18 | − | − |
| 5. | 16 | + | + |
| 6. | 27 | − | − |
| 7. | | − | − |
| 8. | | − | − |
| 9. | | − | − |
| 10. | | − | + |
| 11. | | − | − |
| 12. | | − | − |
| mean | 21.8* | 17%* | 25%* |
| SD | 4.5 | | |
| SE | 1.8 | | |

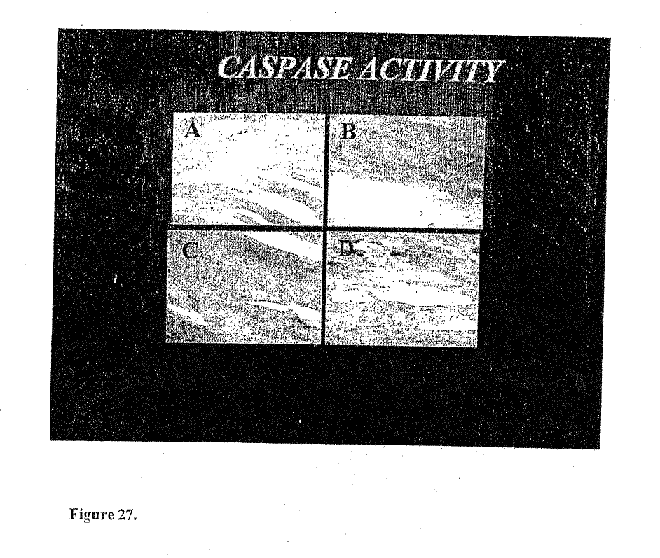

FIG. 27 shows caspase activities (caspase III) in hearts subjected to ischemia/reperfusion and obtained from rats treated with sour cherry seed extract (fraction II) for 14 days. Caspase activity, using immunohistochemistry, was reduced in treated subjects indicating by a reduction in brown color intensity. A: nonischemic aerobically perfused heart; B: drug-free heart subjected to 30 min ischemia followed by 120 min of reperfusion; C and D: rats were treated with 10 mg/kg and 30 mg/kg of sour cherry seed extract (fractions IIa and IIb together) for 14 days, respectively, than hearts were subjected to 30 min ischemia followed by 120 min reperfusion.

The reduction in the infarct size (FIG. 1 and Table 1), the incidence of VF and VT (Table 1), and the caspase activities (FIG. 27) reflected in the “dose-response” postischemic recovery of cardiac function including CF, AF, and LVDP. Thus, lower concentrations (1 mg/kg and 5 mg/kg) of sour cherry seed extract (fractions IIa and IIb together) failed to significantly improve postischemic cardiac function (Table 2b and Table 2c) in comparison with the drug-free control values (Table 2a). However, the higher doses of sour cherry seed extract (10 mg/kg and 30 mg/kg) significantly improved postischemic recovery in CF, AF, and LVDP (Table 2d and Table 2e). These tables (Table 2a to Table 2e), beside the mean, SD, and SEM, show the individual values of HR, CF, AF, an LVDP in each heart, in each untreated and treated group

IN SUMMARY, the patent includes the pharmacological effects of the composition of the following components of ‘O’ phase and solid fraction (fraction II):

The oil (O) phase for ointment production in order to improve vascular circulation and prevention of arteriosclerosis. The following components of sour cherry seed extract (‘O’ fraction) are patented: unsaturated triglyceride components, free fatty acids such as ω-3 α-linoleic acid, hexa-, hepta-, octadecanoic acids, and aldehydes (e.g., hexanal and decadienal), and vitamin E and its isomers (δ-tocopherol, α-tocopherol, and δ-tocotrienol). The α-tocopherol content is 52-53 mg/100 g, while the total tocopherol content is about 80-85 mg/100 g. It is also possible (at the moment no evidence) that some stable prostaglandin derivatives are also responsible for the protective effects of the ‘O’ phase.

The solid phase (phase II) for capsule or tablet production in order to improve vascular circulation and improve ischemia-induced damage in the myocardium. The following components of sour cherry seed extract (fraction II), as major components are patented:

Rhamnetin, malvidin, delfinidin, pinocembrin, naringenin, quercetin, rezveratrol, kaempherol, dihydroquercetin, peonidin, apigenin, pro- and athocyanidins, stilbenes, catechins, gallic acid, gallocatechins, and other antioxidants (e.g., gallotannin).

TABLE 2a
Cardiac function before ISA and after RE in control ischemic/reperfused rat hearts.

| No. of | Before ISA | | | | After 30 min RE | | | | After 60 min RE | | | | After 120 min RE | | | |
|---|---|---|---|---|---|---|---|---|---|---|---|---|---|---|---|---|
| Heart | HR | CF | AF | LVDP | HR | CF | AF | LVDP | HR | CF | AF | LVDP | HR | CF | AF | LVDP |
| 1 | 310 | 31.0 | 44 | 17.8 | 280 | 17 | 8 | 9.6 | 290 | 19 | 7 | 11 | 285 | 18 | 8 | 9.7 |
| 2 | 290 | 24.0 | 51 | 18.7 | 270 | 19 | 14 | 11 | 275 | 18 | 12 | 11.3 | 270 | 19 | 11 | 11.1 |
| 3 | 340 | 23 | 48 | 16.6 | 310 | 14 | 10 | 8.4 | 285 | 17 | 9 | 9.5 | 280 | 16 | 7 | 8.9 |
| 4 | 310 | 28.0 | 57 | 18.2 | 265 | 20 | 11 | 12 | 270 | 21 | 11 | 10.5 | 270 | 19 | 13 | 10 |
| 5 | 300 | 27 | 50 | 17.9 | 295 | 16 | 7 | 9.2 | 290 | 17 | 8 | 9.7 | 290 | 17 | 9 | 9.9 |
| 6 | 285 | 26 | 52 | 17.2 | 280 | 15 | 9 | 9.6 | 275 | 15 | 10 | 10.0 | 280 | 16 | 11 | 8.5 |
| Mean | 306 | 26.5 | 50.3 | 17.7 | 283 | 16.8 | 9.8 | 10.0 | 281 | 17.8 | 9.5 | 10.3 | 279 | 17.5 | 9.8 | 9.7 |
| SD | 20 | 2.6 | 3.9 | 0.7 | 17 | 2.1 | 2.3 | 1.2 | 9 | 1.9 | 1.7 | 0.7 | 7 | 1.3 | 2 | 0.8 |
| SE | 8 | 1.1 | 1.6 | 0.3 | 7 | 0.9 | 0.9 | 0.5 | 4 | 0.8 | 0.7 | 0.3 | 3 | 0.5 | 0.8 | 0.3 |

n = 6 in each group;
heart rate (HR) beats/min;
coronary flow (CF) ml/min;
Aortic flow (AF) ml/min;
left ventricular developed pressure (LVDP) kPa;
ischemia (ISA);
reperfusion (RE).

TABLE 2b
Cardiac function in sour cherry seed extract (fractions IIa and IIb together) treated myocardium, 14
days pretreatment with a daily dose of 1 mg/kg (rat).

| No. of | Before ISA | | | | After 30 min RE | | | | After 60 min RE | | | | After 120 min RE | | | |
|---|---|---|---|---|---|---|---|---|---|---|---|---|---|---|---|---|
| Heart | HR | CF | AF | LVDP | HR | CF | AF | LVDP | HR | CF | AF | LVDP | HR | CF | AF | LVDP |
| 1 | 320 | 28 | 46 | 18.7 | 310 | 16 | 7 | 8.4 | 305 | 15 | 8 | 7.5 | 300 | 16 | 9 | 9.0 |
| 2 | 295 | 27 | 53 | 16.4 | 270 | 17 | 13 | 10.7 | 275 | 14 | 12 | 10.4 | 280 | 16 | 11 | 11.0 |
| 3 | 330 | 31 | 52 | 17.8 | 300 | 15 | 10 | 11.3 | 290 | 19 | 11 | 9.6 | 295 | 18 | 13 | 9.1 |
| 4 | 300 | 24 | 57 | 16.6 | 295 | 16 | 9 | 12.0 | 300 | 13 | 9 | 11.0 | 305 | 14 | 10 | 10.5 |
| 5 | 310 | 26 | 47 | 17.5 | 280 | 18 | 8 | 9.2 | 270 | 18 | 8 | 10.2 | 270 | 17 | 8 | 11.2 |
| 6 | 290 | 29 | 49 | 18.0 | 295 | 17 | 12 | 10 | 285 | 17 | 11 | 9.8 | 275 | 18 | 12 | 10.3 |
| Mean | 308 | 27.5 | 51 | 17.5 | 292 | 16.5 | 9.8 | 10.3 | 288 | 16.0 | 9.8 | 9.8 | 288 | 16.5 | 10.5 | 10.2 |
| SD | 14 | 2.2 | 4 | 0.8 | 13 | 1.0 | 2.1 | 1.2 | 11 | 2.2 | 1.6 | 1.1 | 13 | 1.4 | 1.7 | 0.9 |
| SE | 6 | 0.9 | 1.5 | 0.3 | 5 | 0.4 | 0.9 | 0.5 | 5 | 0.9 | 0.6 | 0.4 | 5 | 0.6 | 0.7 | 0.3 |

n = 6 in each group;
heart rate (HR) beats/min;
coronary flow (CF) ml/min;
aortic flow (AF) ml/min;
left ventricular developed pressure (LVDP) kPa;
ischemia (ISA);
reperfusion (RE).

TABLE 2c
Cardiac function in sour cherry seed extract (fractions IIa and IIb together) treated myocardium,
14 days pretreatment with a daily dose of 5 mg/kg (rat).

| No. of | Before ISA | | | | After 30 min RE | | | | After 60 min RE | | | | After 120 min RE | | | |
|---|---|---|---|---|---|---|---|---|---|---|---|---|---|---|---|---|
| Heart | HR | CF | AF | LVDP | HR | CF | AF | LVDP | HR | CF | AF | LVDP | HR | CF | AF | LVDP |
| 1 | 290 | 27 | 56 | 17.3 | 260 | 19 | 10.0 | 10.5 | 250 | 20 | 11.0 | 10.5 | 260 | 20 | 10.0 | 11.00 |
| 2 | 340 | 32 | 54 | 18.4 | 310 | 20 | 9.0 | 12.4 | 300 | 19 | 9.0 | 11.0 | 290 | 17 | 8.5 | 11.8 |
| 3 | 320 | 24 | 48 | 16.5 | 295 | 16 | 8.5 | 13.0 | 300 | 17 | 9.0 | 10.0 | 320 | 18 | 9.0 | 9.6 |
| 4 | 300 | 23 | 49 | 17.2 | 280 | 15 | 11.9 | 9.5 | 290 | 16 | 12.9 | 9.4 | 280 | 17 | 12.4 | 10.8 |
| 5 | 315 | 27 | 51 | 17.6 | 300 | 17 | 10.9 | 10.8 | 300 | 18 | 12.0 | 11.9 | 300 | 18 | 11 | 12.4 |
| 6 | 330 | 28 | 52 | 17.0 | 305 | 18 | 11.8 | 11.7 | 295 | 18 | 11.5 | 11.3 | 300 | 19 | 12.0 | 10.7 |
| Mean | 321 | 26.8 | 51.7 | 17.3 | 292 | 17.5 | 10.4 | 11.3 | 289 | 18.0 | 10.9 | 10.7 | 292 | 18.2 | 10.5 | 11.1 |
| SD | 20 | 2.9 | 2.7 | 0.6 | 17 | 1.7 | 1.3 | 1.2 | 18 | 1.3 | 1.5 | 0.8 | 19 | 1.1 | 1.5 | 0.9 |
| SE | 8 | 1.2 | 1.1 | 0.2 | 7 | 0.7 | 0.5 | 0.5 | 7 | 0.5 | 0.6 | 0.3 | 8 | 0.4 | 0.6 | 0.4 |

n = 6 in each group;
heart rate (HR) beats/min;
coronary flow (CF) ml/min;
Aortic flow (AF) ml/min;
left ventricular developed pressure (LVDP) kPa;
ischemia (ISA);
reperfusion (RE).

TABLE 2d
Cardiac function in sour cherry seed extract (fractions IIa and IIb together) treated myocardium,
14 days pretreatment with a daily dose of 10 mg/kg (rat).

| No. of | Before ISA | | | | After 30 min RE | | | | After 60 min RE | | | | After 120 min RE | | | |
|---|---|---|---|---|---|---|---|---|---|---|---|---|---|---|---|---|
| Heart | HR | CF | AF | LVDP | HR | CF | AF | LVDP | HR | CF | AF | LVDP | HR | CF | AF | LVDP |
| 1 | 295 | 28 | 46 | 18.2 | 270 | 22.0 | 14.0 | 11.5 | 280 | 24 | 18 | 11.7 | 290 | 25 | 19.0 | 11.2 |
| 2 | 330 | 24 | 55 | 16.4 | 300 | 18.0 | 26.0 | 13.6 | 310 | 19 | 25.0 | 13.2 | 310 | 19 | 24 | 13.0 |
| 3 | 300 | 33 | 52 | 17.6 | 260 | 27 | 21 | 11.4 | 270 | 27 | 24.0 | 12.5 | 300 | 26 | 25.0 | 12.5 |
| 4 | 320 | 27 | 48 | 17.3 | 310 | 22 | 20 | 14.8 | 320 | 24 | 27 | 14.6 | 310 | 23 | 22 | 14.0 |
| 5 | 315 | 26 | 53 | 16.0 | 300 | 21 | 30 | 12.8 | 290 | 22 | 21.0 | 13.5 | 285 | 21 | 22 | 13.5 |
| 6 | 320 | 29 | 51 | 17.9 | 290 | 23 | 15 | 14.7 | 295 | 23 | 17 | 14.7 | 285 | 22 | 18.0 | 14.1 |
| | | | | | | * | * | * | | * | * | * | | * | * | * |
| Mean | 313 | 27.8 | 50.8 | 17.2 | 288 | 22.0 | 21.0 | 13.1 | 294 | 23.2 | 22.0 | 13.4 | 297 | 22.7 | 21.7 | 13.4 |
| SD | 12 | 2.8 | 3.0 | 0.8 | 18 | 2.7 | 5.7 | 1.4 | 17 | 2.4 | 3.7 | 1.1 | 11 | 2.4 | 2.5 | 1.1 |
| SE | 5 | 1.1 | 1.2 | 0.3 | 7 | 1.1 | 2.3 | 0.6 | 7 | 1 | 1.5 | 0.4 | 4 | 1 | 1 | 0.4 |

n = 6 in each group;
heart rate (HR) beats/min;
coronary flow (CF) ml/min;
Aortic flow (AF) ml/min;
left ventricular developed pressure (LVDP) kPa;
ischemia (ISA);
reperfusion (RE).

TABLE 2e
Cardiac function in sour cherry seed extract (fractions IIa and IIb together) treated myocardium,
14 days pretreatment with a daily dose of 30 mg/kg (rat).

| No. of | Before ISA | | | | After 30 min RE | | | | After 60 min RE | | | | After 120 min RE | | | |
|---|---|---|---|---|---|---|---|---|---|---|---|---|---|---|---|---|
| Heart | HR | CF | AF | LVDP | HR | CF | AF | LVDP | HR | CF | AF | LVDP | HR | CF | AF | LVDP |
| 1 | 310 | 24 | 48 | 16.5 | 265 | 21 | 26.0 | 12.8 | 270 | 22 | 28 | 13.6 | 270 | 22 | 28.0 | 13.2 |
| 2 | 290 | 28 | 53 | 18.2 | 255 | 25 | 21.0 | 14.9 | 270 | 25 | 22 | 14.9 | 280 | 23 | 21 | 14.0 |
| 3 | 280 | 33 | 47 | 17.4 | 270 | 28 | 32 | 13.5 | 285 | 28 | 32.0 | 14.5 | 280 | 26 | 30.0 | 14.5 |
| 4 | 295 | 30 | 57 | 17.7 | 290 | 27 | 34 | 15.7 | 290 | 26 | 35 | 16.1 | 290 | 26 | 26 | 15.2 |
| 5 | 330 | 27 | 52 | 16.4 | 310 | 25 | 19 | 12.5 | 300 | 26 | 20.0 | 13.0 | 320 | 25 | 22 | 12.2 |
| 6 | 320 | 28 | 53 | 17.5 | 310 | 23 | 24 | 15.1 | 305 | 27 | 26 | 15.8 | 310 | 27 | 25.0 | 15.1 |
| | | | | | | * | * | * | | * | * | * | | * | * | * |
| | 304 | 28.3 | 51.7 | 17.3 | 283 | 24.8 | 26.0 | 14.1 | 287 | 25.7 | 27.2 | 14.7 | 292 | 24.8 | 25.3 | 14.0 |
| SD | 17 | 2.7 | 3.3 | 0.6 | 22 | 2.3 | 5.4 | 1.2 | 13 | 1.9 | 5.2 | 1.1 | 18 | 1.8 | 3.1 | 1.1 |
| SE | 7 | 1.1 | 1.4 | 0.3 | 9 | 1 | 2.2 | 0.5 | 4 | 0.8 | 2.1 | 0.5 | 7 | 0.7 | 1.3 | 0.4 |

n = 6 in each group;
heart rate (HR) beats/min;
coronary flow (CF) ml/min;
aortic flow (AF) ml/min;
left ventricular developed presure (LVDP) kPa;
ischemia (ISA);
reperfusion (RE).

The above results clearly show that the oil phase and solid phase of the sour cherry seed possess a high cardioprotective effect.

The oil phase is suitable for preparing ointments, preventing stenosis and improvement of circulation. The present invention encompasses the potential active ingredients selected from the group consisting of unsaturated triglyceride components; free fatty acids, e.g. ω-3 α-linolenic acid, hexa-, hepta and octadecanoic acid; and aldehydes (for example hexanal and decadienal), further vitamin E and its isomers (δ-tocopherol, α-tocopherol and δ-tocotrienol). The α-tocopherol content is 52-53 mg/100 g sour cherry seed, while the whole tocopherol content is approximately 80-85 mg/100 g sour cherry seed. It is believed (not proven) that some stable prostaglandin derivative also contributes to the protective effect of the oil fraction of the sour cherry seed.

The solid phase is suitable for improving circulation and reducing ischemic-induced myocardial damages. The present invention encompasses the potential active ingredients selected from the group consisting of rhamnetin, malvidin, delfinidin, pinocembrin, naringenin, quercetin, rezveratrol, kaempherol, dihydroquercetin, peonidin, apigenin, pro- and athocianidines, stilbenes, catechines, gallic acid, gallocatechines and other antioxidants (for example gallotannin).

The solid phase can also be combined with Ca-channel blockers and beta-blockers for use in connection with the indications mentioned above. Such combinations are particularly advantageous, as lower doses are possible which contribute to avoid undesired side effects caused by Ca antagonists and beta blockers.

### REFERENCES

Pearson H. The heart of the matter. Nature Medicine, 2004, 10: 445-446.
Ackerman M J. Cardiac channelopathies: it's in the genes. Nature Medicine, 2004, 10: 463-464.
Tosaki A, Braquet P. DMPO and reperfusion injury: arrhythmia, heart function, electron spin resonance, and nuclear magnetic resonance studies in isolated working guinea pig hearts. Am Heart J, 1990, 120:819-30.
Bak I, Szendrei L, Turoczi T, Papp G, Joo F, Das D K, de Leiris J, Der P, Juhasz B, Varga E, Bacskay I, Balla J, Kovacs P, Tosaki A. Heme oxygenase-1 related carbon monoxide production and ventricular fibrillation in isolated ischemic/reperfused mouse myocardium. FASEB J, 2003, 17: 2133-2135.
Dickson W E, Blehar D J, Carraway R E, Heard S O, Steinberg G, Przyklenk K. Naloxone blocks transferred preconditioning in isolated rabbit hearts. J Mol Cell Cardiol, 2001, 33: 1751-1756.
Schultz J E, Yao Z, Cavero I, Gross G J. Glibenclamide-induced blockade of ischemic preconditioning is time dependent in intact rat heart. Am J Physiol, 1997, 272: H2607-H2615.
Hatip-Al-Khatib I, Iwasaki K, Chung E H, Egashira N, Mishima K, Fujiwara M. Inhibition of poly (ADP-ribose) polymerase and caspase-3, but not caspase-1, prevents apoptosis and improves spatial memory of rats with twice-repeated cerebral ischemia 2004, Life Sci 75:1967-68.
Hancock M B. Visualization of peptide-immunoreactive processes on serotonin-immunoreactive cells using two-color immunoperoxidase staining. J Histochem Cytochem 1984, 32:311-4.

1. A method of improving circulation, preventing stenosis, ameliorating ischemia-induced myocardial damage, and/or providing a cardioprotective effect,
comprising administering, to a subject in need thereof, an amount, effective for such purpose, of a pharmaceutical composition comprising at least one sour cherry fraction, said fraction comprising peonidin, quercetin and procyanocidin.

2. The method of claim 1, said fraction being obtainable as the solid fraction II remaining after extraction of dewalled sour cherry seeds with n-hexane, or obtainable as an extract of said solid fraction II.

3. The method of claim 2, said fraction being a fraction IIa extract obtainable by extraction from the solid fraction II with 70% methanol.

4. The method of claim 2, said fraction being a fraction IIb extract obtainable by extraction from the solid fraction II with a mixture of methanol and hydrochloric acid (9:1).

5. The method of claim 1, wherein said fraction further comprises flavonoids, di-hydro-p-cumaric acid, ferrulic acid, caffeic acid, cyanidin, anthocyan and proanthocyan.

6. The method of claim 5, wherein said fraction further comprises vitamin E-like compounds.

7. The method of claim 1, wherein said fraction further comprises quercetin-3-glucoside, chlorogen acid, gallic acid, epicatechin-3-gallate dimer, acetylated catechin-3-glucoside, p-cumarin acid, and galansin.

8. The method of claim 7, wherein said fraction further comprises ester components, amygdaline and polyphenols.

9. The method of claim 2, said fraction being obtainable by a process comprising the steps of
i) removing the wall of the seed and grinding the inner content of the seed,
ii) extracting the dry grist substance of step i),
iii) drying and filtering the extract obtained in step ii),
iv) extracting the solid fraction obtained in step iii), and
v) evaporating the extract obtained in step iv).

10. The method of claim 1 which is a method of improving circulation.

11. The method of claim 1 which is a method of preventing stenosis.

12. The method of claim 1 which is a method of ameliorating ischemia-induced myocardial damage.

13. The method of claim 1 which is a method of providing a cardioprotective effect.

14. The method of claim 2, said composition comprising both a first extract which is a fraction IIa obtainable by extraction with 70% methanol from said solid, fraction II, and a second extract which is a fraction IIb obtainable by extraction with a mixture of methanol and hydrochloric acid, (9:1) from solid fraction II.

15. The method of claim 1, said extract being obtained as or from the solid fraction II remaining after extraction of sour cherry seeds with n-hexane.

16. The composition of claim 1, said extract lacking, or comprising only a reduced amount relative to the amount in dewalled sour cherry seeds, of at least one compound extractable from dewalled sour cherry seeds by extraction with n-hexane.

17. The method of claim 1, said composition being substantially free of at least one component of dewalled sour cherry seeds that is extractable with n-hexane.

18. The method of claim 1, said composition being substantially free of substantially all components of dewalled sour cherry seeds extractable with n-hexane.

19. The method of claim 1, wherein said fraction has a total tocopherol content of less than 80 mg/100 g, and an alpha-tocopherol content less than 52 mg/100 g.

20. The method of claim 1, wherein said fraction has a total tocopherol concentration substantially less than that of dewalled sour cherry seeds.

21. The method of claim 1, wherein said fraction has an alpha tocopherol concentration substantially less than that of dewalled sour cherry seeds.

22. A method of improving circulation, preventing stenosis, ameliorating ischemia-induced myocardial damage, and/or providing a cardioprotective effect,
comprising administering, to a subject in need thereof, an amount, effective for such purpose, of a pharmaceutical composition comprising at least one sour cherry seed fraction, wherein said fraction comprises at least one compound found in dewalled sour cherry seeds but essentially not extracted by extraction with n-hexane.

23. A method of improving circulation, preventing stenosis, ameliorating ischemia-induced myocardial damage, and/or providing a cardioprotective effect,
comprising administering, to a subject in need thereof, an amount, effective for such purpose, of a pharmaceutical composition comprising at least one sour cherry fraction, said fraction comprising at least one organic cyanide component.

24. The method of claim 23, said fraction further comprising at least one flavonoid.

25. The method of claim 23, said fraction further comprising at least one polyphenol.

26. The method of claim 1, wherein said fraction further comprises rhamnetin, malvidin, delfinidin, pinocembrin, narigenin, resveratrol, kaempherol, dihydroquercetin, apigenin, pro- and athocyanidins, stilbenes, catechins, gallic acid, and gallicatechins.

27. The method of claim 1, wherein said composition is substantially free of free fatty acids found in dewalled sour cherry seeds.
